(19) United States
(12) Patent Application Publication
(10) Pub. No.: US 2013/0073315 A1
(43) Pub. Date: Mar. 21, 2013

(54) DATA PROCESSING AND TRANSFERRING METHOD RELATING TO THE PLACEMENT OF SURGICAL IMPLANTS

(71) Applicant: Philip Cardamone, Victoria (AU)

(72) Inventor: Philip Cardamone, Victoria (AU)

(21) Appl. No.: 13/639,188

(22) PCT Filed: Apr. 19, 2010
(86) PCT No.: PCT/EP10/55109

§ 371 Date: Oct. 3, 2012

(51) Int. Cl. G06Q 50/24; A61B 5/00

(52) U.S. Cl. 705/3; 600/407

(57) ABSTRACT

The invention relates to a method for processing and transferring data relating to the placement of a surgical implant, in which: after a surgical implant has been placed and/or positioned on a patient, the alignment of the implant is measured; information on the measured implant alignment is recorded as electronic implantation information data, in particular as an implantation or case report file; the electronic implantation information data, in particular the implantation or case report file, is evaluated in order to verify that a predetermined or target alignment of the implant has been achieved; the verification information data are made available at a warranty and/or insurance supplier's site; and the verification information data are evaluated at the warranty and/or insurance supplier's site, and a warranty and/or insurance report is created.

The present invention relates to a method of processing and transferring data relating to the placement of a surgical implant.

Providing implants for patients generally involves the implant manufacturers, the physicians working with the implants and, where the surgery is not carried out in a conventional manner, the providers of surgical navigation systems which assist the physicians in correctly placing an implant by means of navigational assistance during the surgery.

It is an object of the present invention to co-ordinate generating and exchanging data between these entities in such a way that an optimum reliability and allocation of responsibility for the outcome of the surgery is achieved.

This object is achieved by a method for processing and transferring data relating to the placement of surgical implants in accordance with claim 1. The sub-claims define advantageous embodiments of the invention.

The invention provides a method for processing and transferring data relating to the placement of a surgical implant, in which:
after a surgical implant has been placed and/or positioned on a patient, the alignment of the implant is measured;
information on the measured implant alignment is recorded as electronic implantation information data, in particular as an implantation or case report file;
the electronic implantation information data, in particular the implantation or case report file, is evaluated in order to verify that a predetermined or target alignment of the implant has been achieved;
the verification information data are made available at a warranty and/or insurance supplier's site;
the verification information data are evaluated at the warranty and/or insurance supplier's site, and a warranty and/or insurance report is created.

It should be noted that the method according to the present invention is neither a treatment method nor a surgical method, nor does it comprise any treatment or surgical steps. The preparation and placement of the implant have been completed before the method according to the invention is initiated or performed. Any interactions with the patient which are necessary in order to perform the method of the invention are routine techniques and do not in themselves involve any health risks.

The present invention advantageously enables a warranty and/or insurance based on the surgical alignment of an implant to be offered. Responsibility for the success of a surgical intervention can thus be allocated more freely, and by competent entities, such that a high level of confidence can be created for patients on the one hand and for physicians and navigation/implant providers on the other hand. The invention is applicable to a large variety of implant surgeries, including joints such as hip and knee surgery and also spinal or any other prosthetic surgery (even outside the field of orthopedics) in which an alignment is always dependent on surgical technique, surgical training, etc.

In one embodiment of the present invention, the alignment of the implant is measured directly after the implant has been placed at the implantation site.

The electronic implantation information data, in particular the implantation or case report file, can be transferred in a variety of ways, including being transferred from the implantation site to the warranty and/or insurance supplier's site or to a verification supplier's site for evaluation and verification. This can be achieved by electronic data transfer, in particular via a direct data upload or via an internet connection, to a data storing and/or processing unit of a warranty and/or insurance or verification supplier, in particular to a data server. It can be in a written and/or electronically encoded form.

If the evaluation of the electronic implantation information data shows that the predetermined or target alignment has been achieved, then a warranty and/or insurance report is created which includes the information that a warranty and/or insurance can be offered. lf, conversely, the evaluation shows that the alignment has not been achieved, then the warranty and/or insurance report will of course include the information that a warranty and/or insurance cannot be offered.

In principle, the invention has been completed once a warranty and/or insurance report has been created. In particular embodiments, however, this warranty and/or insurance report can also then be advantageously used or transferred to particular entities. The warranty and/or insurance report or information about its content or result, or a warranty and/or insurance offer, is for example sent back, in particular automatically, to the implantation site by electronic data transfer. This can be performed at any time after the warranty and/or insurance report has been created, for example at a point in time after the surgery has been completed, such that the physician or other designated person will subsequently find the warranty and/or insurance offer or the warranty and/or insurance itself as a message on their office workstation computer. It can also be, in principle, performed automatically at site without sending information to the warranty and/or insurance supplier's site until post surgery. The warranty and/or insurance report, or information about its content or result, or a warranty and/or insurance offer (or the warranty and/or insurance itself) can alternatively be sent back directly after evaluation, in particular immediately and/or during the ongoing implantation procedure, which can then provide reassurance to the operating team.

The method of the invention may include a step wherein, from the warranty and/or insurance supplier's site, information is sent back recommending further position or alignment adjustments necessary in order to meet warranty or insurance specifications or criteria.

The implant can be identified in accordance with the present invention by means of a medical navigation system which processes data ascertained by determining the type or nature or model of the implant using one or more of the following techniques:
direct identification by imaging the implant and performing shape identification;
direct identification by means of an identification tool which is to be contacted with the implant;
direct identification by means of identifying landmarks on the implant;
indirect identification, for example by means of optical or magnetic references or markers or RFID identifiers attached to the implant.

In accordance with particular embodiments of the present invention, the alignment of the implant is measured and verified by means of one or more of the following:
a medical navigation system which processes position data ascertained by a medical tracking system for determining the position and/or change in position of the implant;
a medical imaging technique such as a CT, MR, x-ray or ultrasound imaging technique;
positionally tracking an instrument, in particular a pointer instrument, which contacts and/or interacts with the implant.

A program which, when it is running on a computer or is loaded onto a computer, causes the computer to perform a method as described above or as described below in particular embodiments, is also an aspect of the present invention, as is a computer program storage medium comprising such a computer program. The present disclosure also includes the provision of a system for processing and transferring data relating to the placement of surgical implants, comprising an alignment measuring system for measuring the predetermined or target alignment of a surgical implant after the implant has been placed and/or positioned on a patient. This system would also include data exchange means and data processing means and also data storage means which enable electronic implantation information data to be recorded, in particular as an implantation or case report file, and evaluated in order to verify that a predetermined or target alignment of the implant has been achieved. Data transfer via a wire connection or wireless data transfer can be used to make the verification information available at a warranty and/or insurance supplier's site, where another data processor and data storage means create and in particular permanently store a warranty and/or insurance report. Computers or workstations or data servers can of course be used to perform the method of the invention and/or individual processes within it, and the information can be sent via local or wide area networks or, as mentioned above, the internet.

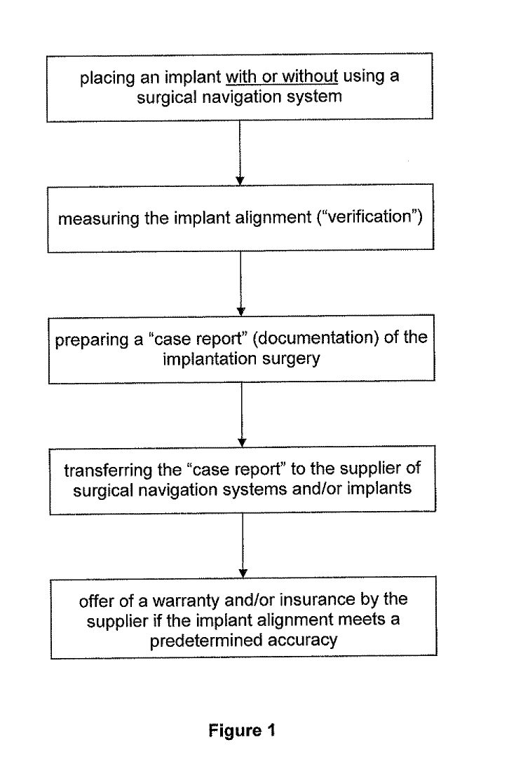

The one attached figure, FIG. 1, shows a flow chart comprising steps which can be performed in connection with the present invention. The flow chart is not exhaustive, i.e. does not show all of the steps which can be performed in a method according to the present invention; conversely, a method in accordance with the invention need not necessarily include all the steps shown in the flow chart and discussed below. For example, the step of placing an implant with or without using a surgical navigation system need not be a step of the method of the invention but can instead be performed beforehand. In general, the following steps are performed in the embodiment of the method according to the flow chart, once the implant has been placed (cf. the comments directly above regarding the first step shown in FIG. 1):
placing an implant with or without using a surgical navigation system;
measuring the implant alignment (“verification”);
preparing a “case report” (documentation) of the implantation surgery;
transferring the “case report” to the supplier of surgical navigation systems and/or implants; and
offer of a warranty and/or insurance by the supplier if the implant alignment meets a predetermined accuracy.

Specifically, the method is performed as described below in accordance with particular embodiments of the invention. It should, however, be noted that the individual features of the present invention as referred to here can be implemented separately or in any expedient combination.

With regard to placing the implant, it is possible to perform implantation surgery for example for the knee (femoral and tibial implants), hip (femoral and/or pelvic implants), shoulder (implants), spine (screws, plates, artificial discs) or trauma applications (screws, plates). Other applications are also conceivable—the invention is applicable to the placement of any surgical implants which have to be placed in a correct surgical position in order to achieve a preferable outcome and a longer service life of the implant, including ophthalmology and the fields of cardiac science, neurosurgery and interventional surgery.

The implant used can exhibit special features in order to allow it to be correctly identified. These features are for example fiducials, markings, landmarks or divots, RFID transponders or special markers on the surface of an implant. It is also possible to not use any special markings and to verify a position using a specially designed verification tool or by optical alignment or some form of imaging modality or a combination of the sources as mentioned above. If fiducials are used, to is possible to use two, three, four or more fiducials in a special arrangement in order to clearly identify the implant.

The implantation surgery can on the one hand be performed with the use of a tracking system, for example an optical (active/passive) or magnetic tracking system which can in turn be linked to a medical navigation system in order to assist a surgeon. One such navigation system (tracking system) is described in DE 196 39 615 A1, and all the features of such a system as described in said document can be used in connection with the present invention and are correspondingly hereby incorporated by reference. On the other hand, any surgery performed using the method of the present invention can also be performed according to its standard technique as applicable.

Different embodiments for measuring the implant alignment (verification) will now be discussed. Such a measurement can use imaging techniques, for example x-ray imaging, CT imaging, fluoroscopy, MRI imaging or ultrasound imaging. Magnetic devices and radio frequencies can be used if the implants contain magnetic and/or RF devices (for example, RFID chips).

An instrument can be used to measure the implant alignment, i.e. to record the positions of divots, fiducials or markings on the implant. If, for example, divots are used on the implant, a surgical pointer (a tracked instrument) can be employed, whereas if RF markers are used on the implant, an RF reader can be used.

Another example of a tracked instrument used as a verification tool would be a tracked cup inserter for a hip implant.

In particular embodiments, specially designed tracked instrument are used to provide accurate information about the position of the implant which would be different depending on the implant used. For this and other embodiments mentioned herein, “tracked” refers to determining a position by means of a medical tracking system which can be connected to a medical navigation system.

The way in which alignment is confirmed can change depending on the different modalities of imaging sources used by a navigation system, such as for example x-ray, CT, MRI, SPEC, PET, ultrasound and/or even optical alignment using light sources of different wavelengths and/or magnetic systems. A combination of any number of these modalities could also be used.

In accordance with another aspect, the implant alignment can be measured using optical light sources which either partially or fully scan the surfaces of the implant and reflect information back to a computer or data processor used in the navigation system. Any light sources provided can be used as an aid to positioning the implant in a correct surgical alignment. It is also possible to use specially designed implants which emit or receive radio frequencies and/or a magnetic field in order to confirm alignment.

Preparing a “case report” of the implantation surgery can also be regarded as a “documentation” of said surgery and can take many forms, for example a written form, an electronic form or an encoded version of written or electronic documents. It can contain details of the patient such as name, age, gender, medical history, etc. It can also contain details of the surgical operation, such as the surgery performed, the implant used, the tracking system used, the date, location and/or duration of the surgery and/or any special incidents during surgery. It can of course also include a general documentation of the steps which would be performed in such surgery.

Details of the implant alignment as measured and verified in accordance with the above description could form part of the case report, and these alignment details could be based either on an anatomical alignment or a biomechanical alignment, depending on the implant used.

The next step, in which the case report is transferred to the supplier of the surgical navigation system and/or the implant supplier, can employ any known data transfer method for supplying such documentation to a receiver in a written and/or electronic format. An upload to a server of the supplier is possible, either via a limited network or via an unlimited network such as the internet. The case report can be transferred by an automated process from the navigation system in situ at the implantation site, once the operation is over and the surgeon is satisfied with the outcome of the surgery. Said case report could be sent automatically, for example via the internet, to the navigation system supplier's company server for an immediate and/or automatic verification process and returned (possibly as soon as it is complete) with a confirmation that the warranty and/or insurance is extended. The last step explained above would already be covered by the last step of the flow chart in FIG. 1, i.e. the offer of a warranty and/or insurance by the implant/navigation supplying company. The warranty and/or insurance would of course only be offered if the implant alignment met a predetermined accuracy, for example if it was confirmed that the implant had been inserted within a particular angular or longitudinal range with respect to a part of a patient's body, for example a femoral bone. The duration of the warranty (fixed for example at 10 or 15 years, or staggered), the scope of the warranty (full or proportionate reimbursement) or other features of the warranty or certain insurance conditions could be made dependent on the level of accuracy determined with respect to the alignment.

1. A method for processing and transferring data relating to the placement of a surgical implant, in which:
after a surgical implant has been placed and/or positioned on a patient, the alignment of the implant is measured;
information on the measured implant alignment is recorded as electronic implantation information data, in particular as an implantation or case report file;
the electronic implantation information data, in particular the implantation or case report file, is evaluated in order to verify that a predetermined or target alignment of the implant has been achieved;
the verification information data are made available at a warranty and/or insurance supplier's site; and
the verification information data are evaluated at the warranty and/or insurance supplier's site, and a warranty and/or insurance report is created.

2. The method according to claim 1, wherein the alignment of the implant is measured directly after the implant has been placed at the implantation site.

3. The method according to claim 1, wherein the electronic implantation information data, in particular the implantation or case report file, is transferred from the implantation site to the warranty and/or insurance supplier's site or to a verification supplier's site for evaluation and verification.

4. The method according to claim 1, wherein the electronic implantation information data, in particular the implantation or case report file, is transferred from the implantation site via electronic data transfer, in particular via a direct data upload or via an internet connection, to a data storing and/or processing unit of a warranty and/or insurance or verification supplier, in particular to a data server.

5. The method according to claim 1, wherein the electronic implantation information data, in particular the implantation or case report file, is in a written and/or electronically encoded form.

6. The method according to claim 1, wherein a warranty and/or insurance report is created which includes the information that a warranty and/or insurance can be offered, if the evaluation of the electronic implantation information data shows that the predetermined or target alignment has been achieved.

7. The method according to claim 1, wherein a warranty and/or insurance report is created which includes the information that a warranty and/or insurance cannot be offered, if the evaluation of the electronic implantation information data shows that the predetermined or target alignment has not been achieved.

8. The method according to claim 1, wherein the warranty and/or insurance report or information about its content or result, or a warranty and/or insurance offer, is sent back, in particular automatically, to the implantation site by electronic data transfer.

9. The method according to claim 1, wherein the warranty and/or insurance report or information about its content or result, or a warranty and/or insurance offer, is sent back, in particular automatically, directly after evaluation, in particular immediately and/or during the ongoing implantation procedure.

10. The method according to claim 1, wherein the implant is identified by means of a medical navigation system which processes data ascertained by determining the type or nature or model of the implant using one or more of the following techniques:
direct identification by imaging the implant and shape identification,
direct identification by means of an identification tool to be contacted with the implant,
direct identification by means of identifying landmarks on the implant,
indirect identification, for example by means of optical or magnetic references or markers, or RFID-identifiers attached to the implant.

11. The method according to claim 1, wherein the alignment of the implant is measured and verified by means of one or more of the following:
a medical navigation system processing position data ascertained by a medical tracking system for determining the position and change in position of the implant,
a medical imaging technique such as a CT, MR, X-Ray or an ultrasound-imaging technique,
positionally tracking an instrument, in particular a pointer instrument, contacting or interacting with the implant.

12. A program which, when it is running on a computer or is loaded onto a computer, causes the computer to perform a method in accordance with claim 1.

13. A computer program storage medium which comprises a computer program according to claim 12.
